(19) United States
(12) Patent Application Publication
(10) Pub. No.: US 2026/0272717 A1
(43) Pub. Date: Sep. 17, 2026

(54) EAR SHAPING KIT

(71) Applicant: Martha Mejia, Doral, FL (US)

(72) Inventor: Martha Mejia, Doral, FL (US)

(21) Appl. No.: 19/077,882

(22) Filed: Mar. 12, 2025

(51) Int. Cl. A61F 11/00; A61F 5/01

(52) U.S. Cl. CPC A61F 11/00; A61F 5/01

(57) ABSTRACT

An ear shaping kit for removing deformities from or otherwise reshaping an auricle includes one or more clip members. Each clip member comprises a front jaw and a back jaw which are movably coupled together via a bias hinge. The back jaw is movable alternately toward and away from the front jaw. The front jaw and the back jaw have engagement surfaces which are configured to clamp an auricle therebetween to urge the auricle toward a desired shape. The bias hinge is coupled to and extends between the front jaw and the back jaw. The bias hinge is configured to bias the back jaw toward the front jaw to clamp the auricle between the back jaw and the front jaw. The bias hinge is also shaped to extend around the helix of the auricle during use.

### (B) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (F) STATEMENT REGARDING PRIOR DISCLOSURES BY THE INVENTOR OR JOINT INVENTOR

Not Applicable

### (G) BACKGROUND OF THE INVENTION

### (1) Field of the Invention

The disclosure relates to ear shaping apparatus and more particularly pertains to a new ear shaping apparatus for removing deformities from or otherwise reshaping an auricle.

### (2) Description of Related Art Including Information Disclosed Under 37 CFR 1.97 and 1.98

Auricular malformations and deformations are changes in shape to an auricle (i.e., the external portion of an ear) due to various sources and can have both functional and psychological effects on those individuals with such deviations in shape. A normally shaped auricle functions to direct noise toward the middle ear, holds up eyeglasses, and secures earbuds. A normally shaped auricle also contributes to a usual self-image such as by symmetrical facial appearance and conformance to normal appearance. Correction of such deviations of shape may therefore aid in restoring function and restoring self-esteem through recreating a normal or typical appearance.

These deviations in shape may be corrected surgically or via various splinting methods using myriad different braces and molding apparatuses. These apparatuses take advantage of the ability of cartilage and other tissues of auricles to be reshaped, especially in infants who have more flexible tissues to reshape. However, existing molding apparatuses use custom-shaped components which must be adjusted by experienced professionals to correctly reshape an auricle. Many auricles fall under one of a number of known common shapes of ears, and a more standardized apparatus or kit is needed to reshape auricles without resorting to devices with custom-made components that require adjustment throughout a course of treatment.

The prior art fails to describe an ear shaping kit which includes one or more clip members that clamp onto an auricle to reshape a selected portion of the auricle via the clamping force molding the auricle into a desired shape. The prior art further fails to describe such a kit in which different clip members are configured to differ from each other in the extents to which they mold the auricle, such that the clip members may be used sequentially to incrementally mold the auricle over a treatment period.

### (H) BRIEF SUMMARY OF THE INVENTION

An embodiment of the disclosure meets the needs presented above by generally comprising a clip member which comprises a front jaw and a back jaw which are movably coupled together via a bias hinge. The back jaw is movable alternately toward and away from the front jaw. The front jaw and the back jaw have engagement surfaces which are configured to clamp an auricle therebetween to urge the auricle toward a desired shape. The bias hinge is coupled to and extends between the front jaw and the back jaw. The bias hinge is configured to bias the back jaw toward the front jaw to clamp the auricle between the back jaw and the front jaw. The bias hinge is also shaped to extend around the helix of the auricle during use.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (I) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective back view of a clip member of an ear shaping kit according to an embodiment of the disclosure.

FIG. 2 is a top view of a clip member of an embodiment of the disclosure.

FIG. 3 is a front view of a clip member of an embodiment of the disclosure.

FIG. 4 is a side view of a clip member of an embodiment of the disclosure.

FIG. 5 is a side kit view of an embodiment of the disclosure, including a plurality of clip members. The back jaws of the clip members are in rest positions as described more fully below in which they are set at predetermined distances from respective front jaws of the respective clip members. The predetermined distances of the clip members are different from each other.

FIG. 6 is an in-use view of an embodiment of the disclosure in which a clip member is clamped onto an auricle of an infant.

FIG. 7 is an in-use view of an embodiment of the disclosure in which a clip member is clamped onto an auricle of an adult.

### (J) DETAILED DESCRIPTION OF THE INVENTION

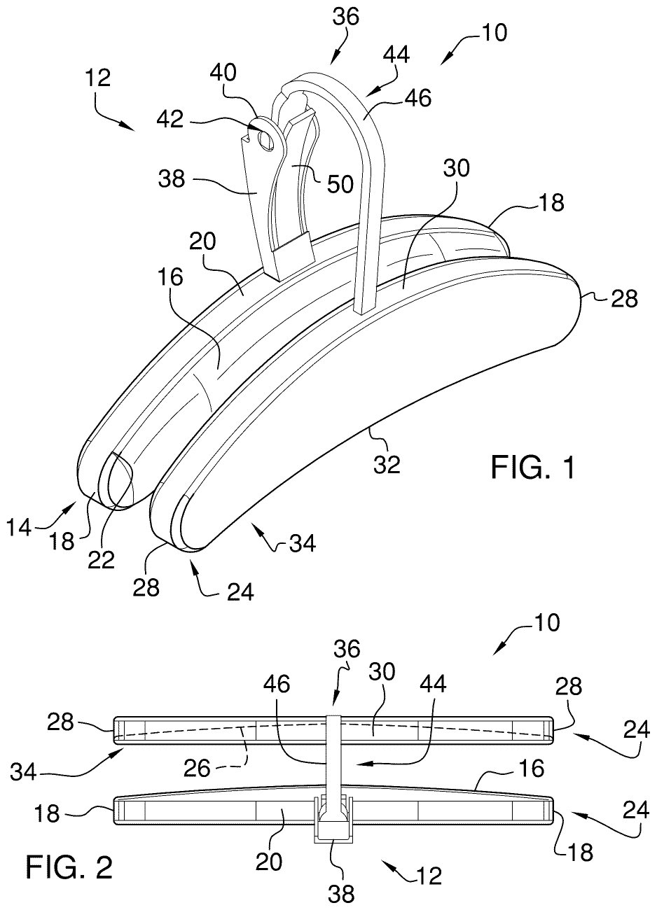

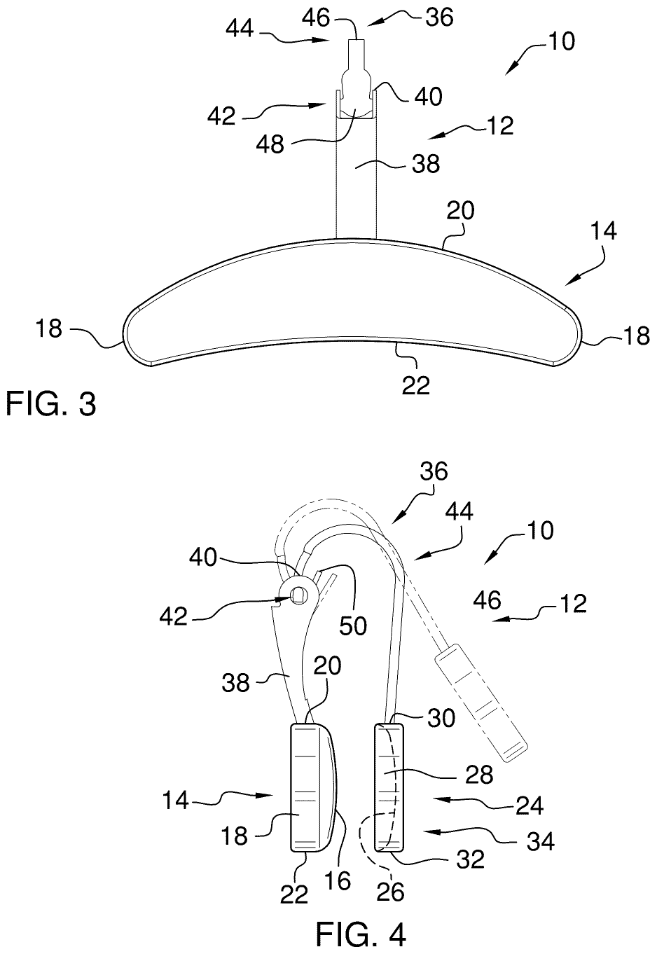

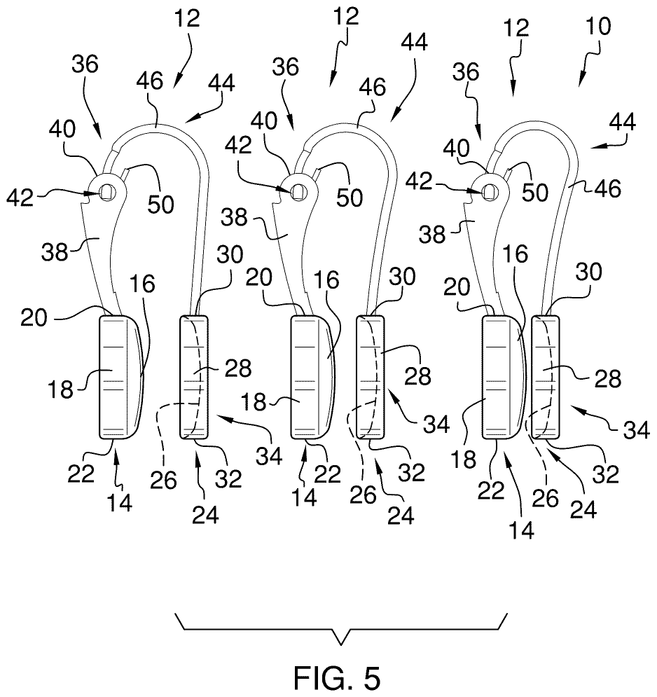

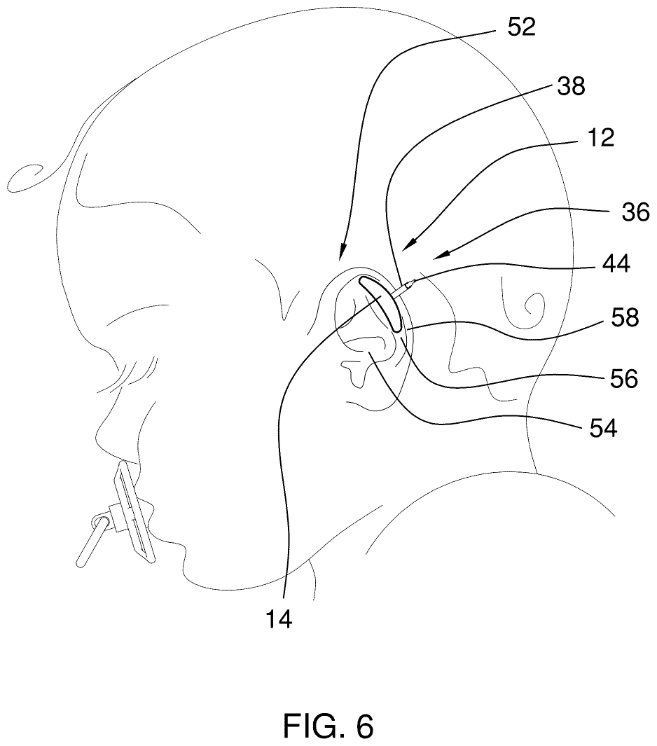

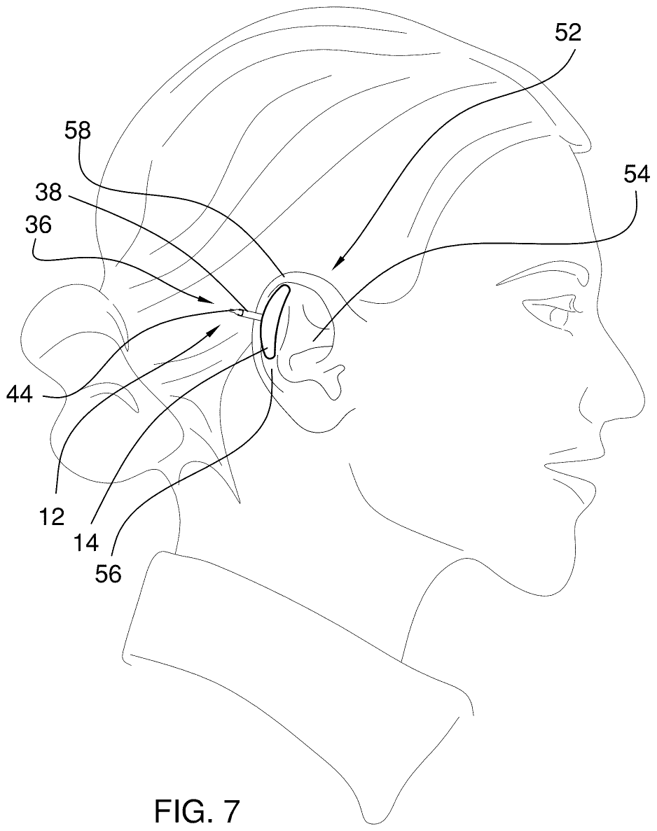

With reference now to the drawings, and in particular to FIGS. 1 through 7 thereof, a new ear shaping apparatus embodying the principles and concepts of an embodiment of the disclosure and generally designated by the reference numeral 10 will be described.

As best illustrated in FIGS. 1 through 7, the ear shaping kit 10 generally comprises a plurality of clip members 12, each of which is configured to secure onto an auricle 52 to mold the auricle 52 into a desired shape. In particular, the clip members 12 are adapted to secure onto a scapha 56 of the auricle 52 adjacent to a helix 58 of the auricle 52 to mold the scapha 56. Each clip member 12 may clamp onto the scapha 56 and the helix 58 simultaneously to also mold the helix 58. Other embodiments may be adapted to mount to and mold other portions of the auricle 52. The clip members 12 are configured to mold the auricle 52 to different thicknesses and may be used sequentially to incrementally mold the auricle 52.

The desired shape may be a shape of the auricle 52 which is beneficial for guiding sound waves toward the associated ear canal and may be a shape which is typical for auricles generally. Said another way, the auricle 52 may be shaped by one or more of the clip members 12 to remove deformities which result in an unnatural or atypical structure. For example, Stahl's ear deformity is a condition in which an extra fold of cartilage develops along the scapha 56 which can occur due to pressures experienced in utero and causes the auricle 52 to be more pointed than what is typical. The desired shape may be a typical shape of the auricle 52 in which the deformity is removed, the scapha 56 being concave on an outward-facing side 54 of the auricle 52, and the helix 58 curling over the scapha 56. In other uses, the desired shape may be a cosmetic change in which the auricle 52 is formed to have some other shape than what is typical, which may be desired by some users.

Each clip member 12 comprises a front jaw 14, a back jaw 24, and a bias hinge 36 coupled to and extending between the front jaw 14 and the back jaw 24. The bias hinge 36 biases the back jaw 24 toward the front jaw 14, at least when positioned in certain locations with respect to the front jaw 14 as further described below. The front jaw 14 and the back jaw 24 have engagement surfaces 16, 26 which are urged toward each other during use via the bias hinge 36 to clamp the auricle 52 therebetween, thereby urging the auricle 52 toward the desired shape.

The engagement surfaces 16, 26 are generally shaped to remove Stahl's ear deformities and similar deviations in the shape of the auricle 52, but the engagement surfaces 16, 26 may have other shapes in other embodiments. In particular, the engagement surface 16 of the front jaw 14 is convex, and the engagement surface 26 of the back jaw 24 is concave and is complementary in shape to the front jaw 14.

The front jaw 14 extends between a pair of ends 18 of the front jaw 14 and has an upper surface 20 and a lower surface 22 which extend between the pair of ends 18 of the front jaw 14. The engagement surface 16 of the front jaw 14 extends between the pair of ends 18 of the front jaw 14 and between the upper surface 20 and the lower surface 22 of the front jaw 14. The front jaw 14 has an arcuate shape wherein the upper surface 20 is convex and the lower surface 22 is concave. The back jaw 24 also extends between a pair of ends 28 of the back jaw 24 and has a top surface 30 and a bottom surface 32 which extend between the pair of ends 28 of the back jaw 24. The engagement surface 26 of the back jaw 24 extends between the pair of ends 28 of the back jaw 24 and between the top surface 30 and the bottom surface 32 of the back jaw 24. The back jaw 24 further has an arcuate shape wherein the top surface 30 is convex and the bottom surface 32 is concave.

The back jaw 24 is positionable in a rest position 34 with respect to the front jaw 14 in which the back jaw 24 is spaced from the front jaw 14 by a predetermined distance. The back jaw 24 is biased toward the rest position 34 by the bias hinge 36 when the back jaw 24 is spaced from the front jaw 14 by a distance greater than the predetermined distance, and the back jaw 24 is unbiased with respect to the front jaw 14 when spaced from the front jaw 14 by a distance less than or equal to the predetermined distance. The predetermined distance is determined such that clamping the clip member 12 onto the auricle 52 will mold the auricle 52 as desired without continuing to urge the back jaw 24 toward the front jaw 14 after reaching the predetermined distance from the front jaw 14, thereby avoiding potential damage to the auricle 52.

The predetermined distances of the plurality of clip members 12 are different from each other. This is so that multiple clip members 12 of the plurality of clip members 12 may be used incrementally in order of greatest predetermined distance to least predetermined distance to steadily mold the auricle 52 over multiple stages. Different starting clip members 12 may be selected depending on a size of the auricle 52 being reshaped and the severity of the deformity. In some cases, only one clip member 12 may be needed to reshape the auricle 52 to the desired shape. Additionally, in some embodiments of the disclosure, only one clip member 12 is provided.

The bias hinge 36 biases the back jaw 24 as described above. The bias hinge 36 is coupled to and extends between the upper surface 20 of the front jaw 14 and the top surface 30 of the back jaw 24. The bias hinge 36 is shaped to extend around the helix 58 of the auricle 52 during use. The bias hinge 36 comprises a front link 38, a back link 44, and a leaf spring 50. The front link 38 is coupled to and extends away from the upper surface 20 of the front jaw 14. The back link 44 is pivotably coupled to the front link 38 at a pivot point 42 which is positioned adjacent to a distal end 40 of the front link 38 with respect to the front jaw 14. The back link 44 comprises a main portion 46 which extends from the pivot point 42 to the top surface 30 of the back jaw 24 and a protrusion portion 48 which is coupled to the main portion 46 at the pivot point 42 and extends away from the main portion 46.

The leaf spring 50 is coupled to the front link 38 adjacent to the front jaw 14 and extends away from the front jaw 14 along the front link 38. The protrusion portion 48 of the back link 44 is positioned to urge the leaf spring 50 against bias when the back jaw 24 is moved away from the front jaw 14 beyond the rest position 34. The protrusion portion 48 of the back link 44 is further positioned to be disengaged from the leaf spring 50 when the back jaw 24 is positioned between the rest position 34 and the front jaw 14. In other embodiments, the bias hinge 36 may comprise other biasing members alternatively to or in addition to the leaf spring 50. Such other biasing members may be positioned and sized to engage the back link 44 to bias it toward the rest position 34 and to disengage from the back jaw 24 when the back jaw 24 is in the rest position 34 or between the front jaw 14 and the rest position 34. The other biasing members may include a torsion spring, a compression spring, a tension spring, an elastic band, or the like.

Other mechanisms which bias the back jaw 24 toward the rest position 34 may be used, including those which cause the back jaw 24 to move linearly rather than pivoting with respect to the front jaw 14. Compression springs, tension springs, elastic bands, or other such biasing members may cause such linear movement of the back jaw 24 with respect to the front jaw 14.

In some embodiments, the bias hinge 36 or another bias mechanism may not disengage from the back jaw 24 such that the back jaw 24 continually experiences a force urging the back jaw 24 toward the front jaw 14. In such embodiments, the bias hinge 36 or other bias mechanism may be configured to decrease a magnitude of the force urging the back jaw 24 toward the front jaw 14 such that the force has a nominal magnitude which does not cause further reshaping of the auricle 52 after the auricle 52 attains an intended shape. In such embodiments, the bias hinge 36s or other bias mechanisms of the different clip members 12 may be configured to clamp the auricle 52 with different magnitudes of force or apply the nominal magnitude at different positions of the back jaw 24 with respect to the front jaw 14 such that different stages of molding may still be achieved.

In use, one or more clip members 12 of the ear shaping kit 10 are selected based on the size of the auricle 52 and the severity of the deformity being treated. Or, in other use cases such as shaping the auricle 52 uniquely for cosmetic purposes, the extent to which the desired shape differs from the initial shape of the auricle 52 may inform the selection of which clip member 12 is selected first. The clip members 12 are interchangeably and incrementally applied to the auricle 52 over a treatment period until the auricle 52 has been formed into the desired shape. Clip members 12 may be interchanged, for example, when the back jaw 24 of the clip member 12 currently mounted to the auricle 52 is at the rest position 34 or when the clip member 12 stops applying enough clamping force to mold the auricle 52.

When a selected clip member 12 is clamped onto the auricle 52, the front jaw 14 is positioned against the scapha 56 of the auricle 52 on the outward-facing side 54 of the auricle 52. The back jaw 24 is positioned on an inward-facing side of the auricle 52 (not shown, facing toward a head of a wearer of the clip member 12). The bias hinge 36 extends around the helix 58 of the auricle 52. So positioned, the selected clip member 12 clamps the scapha 56 between the back jaw 24 and the front jaw 14 to reshape the scapha 56. The selected clip member 12 may also clamp the helix 58 between the back jaw 24 and the front jaw 14 to reshape the helix 58. The back jaw 24 and the front jaw 14 of the selected clip member 12 may be placed on other portions of the auricle 52 in other embodiments which are configured to reshape those portions of the auricle 52.

Different embodiments may comprise clip members 12 with different ranges of predetermined distances. For example, clip members 12 of a kit 10 for use on an infant's auricle may have predetermined distances of 5.0 millimeters, 4.0 millimeters, and 3.0 millimeters, or other increments between 5.0 millimeters and 3.0 millimeters. Clip members 12 of a kit 10 for use on an adult's auricle may have predetermined distances lying in a range of 9.0 millimeters to 5.0 millimeters. The back jaws 24 and the front jaws 14 of different kits 10 may also have different shapes which are adapted for use on auricles having different shapes. These shapes may be selected to adapt to a variety of common shapes of auricles. In some embodiments, the back jaws 24 and the front jaws 14 of the clip members 12 may be custom-formed to adapt to a given auricle to be reshaped. The given auricle may be scanned via a three-dimensional scanner, and the engagement surfaces 26, 16 of the back jaws 24 and the front jaws 14 may be formed to focus pressure on the portion of the auricle to be reshaped.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An ear shaping apparatus comprising:
a clip member comprising:
a front jaw and a back jaw which are movably coupled together, the back jaw being movable alternately toward and away from the front jaw, the front jaw and the back jaw having engagement surfaces which are configured to clamp an auricle therebetween to urge the auricle toward a desired shape; and
a bias hinge coupled to and extending between the front jaw and the back jaw, the bias hinge being configured to bias the back jaw toward the front jaw to clamp the auricle between the back jaw and the front jaw, the bias hinge being shaped and configured to extend around the helix of the auricle.

2. The ear shaping apparatus of claim 1, wherein the back jaw is positionable in a rest position with respect to the front jaw in which the back jaw is spaced from the front jaw by a predetermined distance, the back jaw being biased toward the rest position by the bias hinge when the back jaw is spaced from the front jaw by a distance greater than the predetermined distance, the back jaw being unbiased with respect to the front jaw when spaced from the front jaw by a distance less than or equal to the predetermined distance.

3. The ear shaping apparatus of claim 2, wherein the bias hinge comprises:
a front link coupled to and extending away from the front jaw;
a back link pivotably coupled to the front link at a pivot point which is positioned adjacent to a distal end of the front link with respect to the front jaw, the back link being coupled to the back jaw; and
a leaf spring coupled to the front link adjacent to the front jaw, the leaf spring being positioned such that the back link urges the leaf spring against bias when the back jaw is moved away from the front jaw beyond the rest position, the leaf spring being positioned such that the back link disengages from the leaf spring when the back jaw is positioned between the rest position and the front jaw.

4. The ear shaping apparatus of claim 3, wherein the back link of the bias hinge comprises a main portion which extends from the pivot point to the back jaw, the main portion of the back link being coupled to the back jaw, the back link comprising a protrusion portion which is coupled to the main portion at the pivot point and extends away from the main portion, the protrusion portion being positioned to urge the leaf spring against bias when the back jaw is moved away from the front jaw beyond the rest position, the protrusion portion of the back link being positioned to be disengaged from the leaf spring when the back jaw is positioned between the rest position and the front jaw.

5. The ear shaping apparatus of claim 1, wherein the engagement surface of the front jaw is convex, the engagement surface of the back jaw being concave and being complementary in shape to the front jaw.

6. The ear shaping apparatus of claim 1, wherein the front jaw is shaped and configured to be positioned against a scapha of the auricle adjacent to a helix of the auricle.

7. The ear shaping apparatus of claim 1, wherein:
the front jaw extends between a pair of ends of the front jaw, the front jaw having an upper surface and a lower surface which extend between the pair of ends of the front jaw, the engagement surface of the front jaw extending between the pair of ends of the front jaw and between the upper surface and the lower surface of the front jaw, the front jaw having an arcuate shape wherein the upper surface is convex and the lower surface is concave; and
the back jaw extends between a pair of ends of the back jaw, the back jaw having a top surface and a bottom surface which extend between the pair of ends of the back jaw, the engagement surface of the back jaw extending between the pair of ends of the back jaw and between the top surface and the bottom surface of the back jaw, the back jaw having an arcuate shape wherein the top surface is convex and the bottom surface is concave.

8. An ear shaping apparatus comprising:
a clip member comprising:
a front jaw and a back jaw which are movably coupled together, the back jaw being movable alternately toward and away from the front jaw, the back jaw being positionable in a rest position with respect to the front jaw in which the back jaw is spaced from the front jaw by a predetermined distance, the back jaw being biased toward the rest position when the back jaw is spaced from the front jaw by a distance greater than the predetermined distance, the back jaw being unbiased with respect to the front jaw when spaced from the front jaw by a distance less than or equal to the predetermined distance, the front jaw and the back jaw having engagement surfaces which are configured to clamp an auricle therebetween to urge the auricle toward a desired shape, the engagement surface of the front jaw being convex, the engagement surface of the back jaw being concave and being complementary in shape to the front jaw, the front jaw being shaped and configured to be positioned against a scapha of the auricle adjacent to a helix of the auricle;
wherein the front jaw extends between a pair of ends of the front jaw, the front jaw having an upper surface and a lower surface which extend between the pair of ends of the front jaw, the engagement surface of the front jaw extending between the pair of ends of the front jaw and between the upper surface and the lower surface of the front jaw, the front jaw having an arcuate shape wherein the upper surface is convex and the lower surface is concave;
wherein the back jaw extends between a pair of ends of the back jaw, the back jaw having a top surface and a bottom surface which extend between the pair of ends of the back jaw, the engagement surface of the back jaw extending between the pair of ends of the back jaw and between the top surface and the bottom surface of the back jaw, the back jaw having an arcuate shape wherein the top surface is convex and the bottom surface is concave; and
a bias hinge coupled to and extending between the front jaw and the back jaw, the bias hinge biasing the back jaw toward the rest position when the back jaw is spaced from the front jaw by a distance greater than the predetermined distance, the bias hinge being shaped and configured to extend around the helix of the auricle, the bias hinge comprising:
a front link coupled to and extending away from the front jaw, the front link being positioned on the upper surface of the front jaw;
a back link pivotably coupled to the front link at a pivot point which is positioned adjacent to a distal end of the front link with respect to the front jaw, the back link comprising a main portion which extends from the pivot point to the back jaw, the main portion of the back link being coupled to the top surface of the back jaw, the back link comprising a protrusion portion which is coupled to the main portion at the pivot point and extends away from the main portion; and
a leaf spring coupled to the front link adjacent to the front jaw, the leaf spring extending away from the front jaw along the front link, the protrusion portion of the back link being positioned to urge the leaf spring against bias when the back jaw is moved away from the front jaw beyond the rest position, the protrusion portion of the back link being positioned to be disengaged from the leaf spring when the back jaw is positioned between the rest position and the front jaw.

9. An ear shaping kit comprising:
a plurality of clip members, each clip member of the plurality of clip members comprising:
a front jaw and a back jaw which are movably coupled together, the back jaw being movable alternately toward and away from the front jaw, the front jaw and the back jaw having engagement surfaces which are configured to clamp an auricle therebetween to urge the auricle toward a desired shape; and
a bias hinge coupled to and extending between the front jaw and the back jaw, the bias hinge being configured to bias the back jaw toward the front jaw to clamp the auricle between the back jaw and the front jaw, the bias hinge being shaped and configured to extend around the helix of the auricle; and
wherein the clip members of the plurality of clip members are configured to clamp the auricle differently to shape the auricle to different thicknesses.

10. The ear shaping kit of claim 9, wherein:
for each clip member of the plurality of clip members:
the back jaw is positionable in a rest position with respect to the front jaw in which the back jaw is spaced from the front jaw by a predetermined distance, the back jaw being biased toward the rest position by the bias hinge when the back jaw is spaced from the front jaw by a distance greater than the predetermined distance, the back jaw being unbiased with respect to the front jaw when spaced from the front jaw by a distance less than or equal to the predetermined distance; and
the predetermined distances of the plurality of clip members are different from each other.

11. The ear shaping kit of claim 9, wherein the bias hinge of each clip member comprises:
a front link coupled to and extending away from the front jaw;
a back link pivotably coupled to the front link at a pivot point which is positioned adjacent to a distal end of the front link with respect to the front jaw, the back link being coupled to the back jaw; and
a leaf spring coupled to the front link adjacent to the front jaw, the leaf spring being positioned such that the back link urges the leaf spring against bias when the back jaw is moved away from the front jaw beyond the rest position, the leaf spring being positioned such that the back link disengages from the leaf spring when the back jaw is positioned between the rest position and the front jaw.

12. The ear shaping kit of claim 11, wherein the back link of the bias hinge of each clip member comprises a main portion which extends from the pivot point to the back jaw, the main portion of the back link being coupled to the back jaw, the back link comprising a protrusion portion which is coupled to the main portion at the pivot point and extends away from the main portion, the protrusion portion being positioned to urge the leaf spring against bias when the back jaw is moved away from the front jaw beyond the rest position, the protrusion portion of the back link being positioned to be disengaged from the leaf spring when the back jaw is positioned between the rest position and the front jaw.

13. The ear shaping kit of claim 9, wherein the engagement surface of the front jaw of the bias hinge of each clip member is convex, the engagement surface of the back jaw being concave and being complementary in shape to the front jaw.

14. The ear shaping kit of claim 9, wherein the front jaw of the bias hinge of each clip member is shaped and configured to be positioned against a scapha of the auricle adjacent to a helix of the auricle.

15. The ear shaping kit of claim 9, wherein, for the bias hinge of each clip member:
the front jaw extends between a pair of ends of the front jaw, the front jaw having an upper surface and a lower surface which extend between the pair of ends of the front jaw, the engagement surface of the front jaw extending between the pair of ends of the front jaw and between the upper surface and the lower surface of the front jaw, the front jaw having an arcuate shape wherein the upper surface is convex and the lower surface is concave; and
the back jaw extends between a pair of ends of the back jaw, the back jaw having a top surface and a bottom surface which extend between the pair of ends of the back jaw, the engagement surface of the back jaw extending between the pair of ends of the back jaw and between the top surface and the bottom surface of the back jaw, the back jaw having an arcuate shape wherein the top surface is convex and the bottom surface is concave.
